(19) United States
(12) Patent Application Publication
(10) Pub. No.: US 2026/0175043 A1
(43) Pub. Date: Jun. 25, 2026

(54) DEVICES AND SYSTEMS FOR CHRONIC OPTICAL NEUROMODULATION

(71) Applicants: Dena SHAHRIARI, Vancouver (CA); Shahriar SHALILEH, Vancouver (CA)

(72) Inventors: Dena SHAHRIARI, Vancouver (CA); Shahriar SHALILEH, Vancouver (CA)

(21) Appl. No.: 18/839,751

(22) PCT Filed: Jun. 8, 2024
(86) PCT No.: PCT/IB2024/055623

§ 371 Date: Aug. 20, 2024

Related U.S. Application Data
(60) Provisional application No. 63/472,443, filed on Jun. 12, 2023.

(51) Int. Cl. A61N 5/06; A61B 18/00; A61M 5/142

(52) U.S. Cl. CPC A61N 5/0622; A61M 5/142; A61N 5/0603; A61N 5/062; A61B 2018/00732; A61B 2018/00761; A61B 2018/00875; A61N 2005/0612; A61N 2005/0626; A61N 2005/0652; A61N 2005/0659; A61N 2005/0661; A61N 2005/0662

(57) ABSTRACT

Disclosed herein is a system for optical stimulation of a target region in a living body. The system includes one or more probes subcutaneously placed adjacent to the target region connected to a microdevice subcutaneously placed in the living body. Each probe includes a biocompatible elastic strip, a flexible-length wire inside the elastic strip, and a plurality of light emitting elements mounted along the wire. The microdevice includes a processing unit connected to one or more light drivers coupled to the plurality of light emitting elements delivering light thereto. The processing unit includes a memory and a processor executing instructions stored in the memory to perform a method of optically stimulating cells of the target region by delivering a light beam with a predetermined set of characteristics to at least one light emitting element utilizing at least one light driver.

### TECHNICAL FIELD

The present disclosure generally relates to optical stimulation of biological cells, and particularly, to a fully implantable system without any tethers or wires in a living body capable of autonomous self-reliant chronic light simulation of target cells of a target tissue or region in the living body.

### BACKGROUND OF THE INVENTION

Optical modulation of cells can be accomplished by, for example, optogenetics in which a targeted group of cells are sensitized to light via genetic modification. Optogenetics has revolutionized global knowledge in the field of brain circuitry since 2004 but its use in other body parts is much less used due to technological limitations.

Commercially available glass fibers have been commonly implanted in animal brains since 2005. Due to the ease of acquiring and implementation of glass fibers, optogenetics has become a go-to-tool in brain research. One of the problems of using glass fibers is inflammation of tissue due to insertion of glass fibers there into. Additionally, commonly used optogenetic methods and devices require keeping a subject (e.g., a rat) in a cage in order to deliver light via external light sources, controlling a stimulation process by external controllers, and recharging a power source of an exemplary device with external charging systems. Use of such confined areas may cause limitation of subjects'movements as well as parameters should be studied during optical stimulation, such as effect of passing time, running, walking, etc. For this reason, there is lack of a device for large animal studies, for example, pigs; which are not being kept in small cages in the animal facilities, and consequently, study of human optogenetics is limited using such devices. Furthermore, application of optogenetics in peripheral nervous system and spinal cord circuitry is yet to be harnessed.

Therefore, there is a need in the art for a device and system being capable of autonomous self-reliant chronic light simulation. An exemplary device and system should be fully implantable in a subject's body; allowing for optical stimulation of the subject in a non-confined environment. Moreover, an exemplary device and system needs to be employed for tether-free behavioral assays, which requires a subject to be placed out of cages in a secondary or free environment.

### SUMMARY OF THE INVENTION

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes a system for optical stimulation of a target region in a living body. In an exemplary embodiment, the system may include one or more probes configured to be put in the vicinity of the target region and a microdevice configured to be subcutaneously placed under skin of the living body. In an exemplary embodiment, each probe of the one or more probes may include an elastic strip including two or more layers of a soft flexible biocompatible material, an electrically conductive wire with a variable length placed between two layers of the two or more layers of the elastic strip, and a plurality of light emitting elements mounted on the wire along the length thereof. In an exemplary embodiment, the microdevice may include a substrate including a piece of a soft flexible biocompatible material, one or more light drivers attached onto the substrate, and a processing unit attached onto the substrate. In an exemplary embodiment, each light driver of the one or more light drivers may be coupled to one or more light emitting elements of the plurality of light emitting elements. In an exemplary embodiment, each light driver may be utilized to deliver light through the one or more light emitting elements of the plurality of light emitting elements.

In an exemplary embodiment, the processing unit may be coupled to the one or more light drivers. In an exemplary embodiment, the processing unit may include a memory having processor-readable instructions stored therein and a processor utilized to access the memory and execute the processor-readable instructions. In an exemplary embodiment, the processor may perform a method by executing the processor-readable instructions. In an exemplary embodiment, the method may include optically stimulating cells of the target region by light delivery with a predetermined set of characteristics through each light emitting elements of the plurality of light emitting elements utilizing the one or more light drivers.

In an exemplary embodiment, light delivery with the predetermined set of characteristics may include light delivery with a predetermined magnitude of at least one of a wavelength of the light beam, a frequency of the light beam, an intensity of the light beam, a time duration of light delivery, and combinations thereof.

In an exemplary embodiment, light delivery with the predetermined set of characteristics may include light delivery with a wavelength in at least one range of a visible range of 380 nm to 750 nm, an ultraviolet (UV) range of 10 nm to 380 nm, an infrared (IR) range of 750 nm to 1 mm, and combinations thereof. In an exemplary embodiment, light delivery with the predetermined set of characteristics may include light delivery with a frequency in a range of 0.001 Hz to 2 MHz. In an exemplary embodiment, light delivery with the predetermined set of characteristics may include light delivery with an intensity in a range of 0 W/cm² to 6.95 W/cm². In an exemplary embodiment, light delivery with the predetermined set of characteristics may include light delivery with a time duration in a range of 0 seconds to one or more months.

In an exemplary embodiment, the elastic strip may include a strip of a soft flexible biocompatible polymer with a length in a range of 0.01 cm to 50 cm and a width in a range of 300 μm to 10 cm. In an exemplary embodiment, each of the wire and the elastic strip may include a stretchable length up to 70 percent of an initial length thereof. In an exemplary embodiment, the wire may include a serpentine-shaped wire with a length in a range of 0.01 cm to 50 cm.

In an exemplary embodiment, each light emitting element of the plurality of light emitting elements may include a light emitting diode (LED). In an exemplary embodiment, each two light emitting elements of the plurality of light emitting elements may be arranged at a location of at least one of a tip of the wire, along the wire, and combinations thereof, in series or parallel relation apart from each other within a distance of more than 100 μm.

In an exemplary embodiment, the substrate may include a piece of a soft flexible biocompatible polymer with a length in a range of 5 mm to 20 mm and a width in a range of 5 mm to 20 mm.

In an exemplary embodiment, the system may further include an electrical sensor attached to the wire. In an exemplary embodiment, the electrical sensor may be utilized to measure an electrical parameter of the target region at least one of before, during, and after optical stimulation of the target region and send the measured electrical parameter to the processing unit. In an exemplary embodiment, the electrical parameter may include at least one of an electrical current of the target region, an electrical voltage of the target region, and combinations thereof. In an exemplary embodiment, the electrical sensor may be coupled to the processing unit/microdevice via at least one of the wire, a wireless connection, and combinations thereof.

In an exemplary embodiment, the microdevice may further include a real-time calendar (RTC) placed on the substrate. In an exemplary embodiment, the RTC may be coupled to the processing unit. In an exemplary embodiment, the method may further include at least one of starting light delivery through each light emitting element of the plurality of light emitting elements at a first predetermined time, ceasing light delivery through each light emitting element of the plurality of light emitting elements at a second predetermined time, turning on one or more functionalities of the microdevice, turning off one or more functionalities of the microdevice, switching to a different predetermined set of characteristics of light delivery at a pre-scheduled time or a time during light delivery, and combinations thereof. In an exemplary embodiment, the method may further include starting light delivery through each light emitting element of the plurality of light emitting elements at a first predetermined time and ceasing light delivery through each light emitting element of the plurality of light emitting elements at a second predetermined time.

In an exemplary embodiment, the system may further include a temperature sensor adhered onto the elastic strip of the probe. In an exemplary embodiment, the temperature sensor may be coupled to the processing unit. In an exemplary embodiment, the method may further include measuring a temperature of the target region at least one of before, during, and after optical stimulation of the target region utilizing the temperature sensor, comparing the measured temperature with a threshold temperature value, and performing one or more processes of a set of processes responsive to the measured temperature being more than the threshold temperature value. In an exemplary embodiment, the set of processes may include changing one or more characteristics of the predetermined set of characteristics and ceasing light delivery through one or more light emitting elements of the plurality of light emitting elements.

In an exemplary embodiment, the microdevice may include an ultra-low energy consuming device with a required power in a range of 360 nW to 160 mW. In an exemplary embodiment, the system may further include a wirelessly power recharging mechanism. In an exemplary embodiment, the wirelessly power recharging mechanism may include a rechargeable battery coupled to the microdevice via a soft stretchable electrically conductive connecting line, a wireless power receiver coupled to the microdevice, a wireless power transmitter including a power generation unit and a transmitter antenna, and a wireless battery charging module attached onto the substrate.

In an exemplary embodiment, the rechargeable battery may be utilized to provide/supply a power of the microdevice. In an exemplary embodiment, the rechargeable battery may be subcutaneously placed under skin of the living body. In an exemplary embodiment, the wireless power receiver may include a receiver antenna connected to the microdevice. In an exemplary embodiment, the receiver antenna may be subcutaneously placed under skin of the living body. In an exemplary embodiment, the transmitter antenna may be wirelessly coupled to the receiver antenna. In an exemplary embodiment, the transmitter antenna may be placed at a location over skin of the living body in the vicinity of the receiver antenna. In an exemplary embodiment, the power generation unit may be placed outside the living body. In an exemplary embodiment, the wireless battery charging module may be coupled to the wireless power receiver and the rechargeable battery. In an exemplary embodiment, the rechargeable battery may be charged by the wireless battery charging module utilizing a power transmitted from the wireless power transmitter to the wireless power receiver at a frequency range of 100 kHz to 200 kHz.

In an exemplary embodiment, the system may further include a temperature sensor adhered onto the substrate of the microdevice. In an exemplary embodiment, the temperature sensor may be coupled to the processing unit. In an exemplary embodiment, the method may further include measuring a temperature of the microdevice during recharging the rechargeable battery utilizing the temperature sensor, comparing the measured temperature with a threshold temperature value, and ceasing recharging of the rechargeable battery responsive to the measured temperature being more than the threshold temperature value.

In an exemplary embodiment, the system may further include a drug delivery mechanism utilized to deliver a drug to the target region. In an exemplary embodiment, the drug delivery mechanism may include a drug delivery channel formed in the elastic strip and a drug delivery pump adhered onto the substrate. In an exemplary embodiment, the drug delivery pump may be coupled to the processing unit. In an exemplary embodiment, the method may further include releasing a drug into the target region through the drug delivery channel utilizing the drug delivery pump.

In an exemplary embodiment, the system may further include at least one of one or more photodetectors mounted on the probe, one or more biomarker sensors mounted on the probe, one or more impedance sensors mounted on the probe, and one or more electrical stimulation electrodes mounted on the probe. In an exemplary embodiment, the one or more photodetectors may be utilized to detect and measure cellular activity of the target region. In an exemplary embodiment, the one or more biomarker sensors may be utilized to sense an antibody in the target region via at least one of fast sensing, short-term sensing, chronic sensing, and combinations thereof. In an exemplary embodiment, the one or more impedance sensors may be utilized to measure at least one of a level of neural myelination, fat formation/insulation, blood flow, and combinations thereof in the target region. In an exemplary embodiment, the one or more electrical stimulation electrodes may be utilized to electrically stimulating cells of the target region.

Other exemplary systems, methods, features and advantages of the implementations will be, or will become, apparent to one of ordinary skill in the art upon examination of the following figures and detailed description. It is intended that all such additional systems, methods, features and advantages be included within this description and this summary, be within the scope of the implementations, and be protected by the claims herein.

### BRIEF DESCRIPTION OF DRAWINGS

The drawing figures depict one or more implementations in accord with the present teachings, by way of example only, not by way of limitation. In the figures, like reference numerals refer to the same or similar elements.

FIG. 1 schematically shows an exemplary system for optical neurostimulation implanted in an exemplary living body, consistent with one or more exemplary embodiments of the present disclosure.

FIG. 2 schematically shows an exemplary probe of one or more exemplary probes, consistent with one or more exemplary embodiments of the present disclosure.

FIG. 3A schematically shows an exemplary system for optical neurostimulation of an exemplary target region in an exemplary living body illustrating a top view of an exemplary microdevice, consistent with one or more exemplary embodiments of the present disclosure.

FIG. 3B schematically shows a bottom view of an exemplary microdevice in connection with exemplary one or more probes, consistent with one or more exemplary embodiments of the present disclosure.

FIG. 4 schematically shows an exemplary block diagram of battery recharging circuit, consistent with one or more exemplary embodiments of the present disclosure.

FIG. 5 shows a high-level functional block diagram of a computer system, consistent with one or more exemplary embodiments of the present disclosure.

FIG. 6 shows a flowchart of an exemplary method for optical stimulation of cells in a living body, consistent with one or more exemplary embodiments of the present disclosure.

FIG. 7 schematically shows an exemplary process of implanting an exemplary probe in the vicinity of spinal cord of an exemplary living body, consistent with one or more exemplary embodiments of the present disclosure.

FIG. 8 shows an exemplary device implantation and an exemplary probe placement in the vicinity of spinal cord of an exemplary rat, consistent with one or more exemplary embodiments of the present disclosure.

FIG. 9 shows Martinez open field behavioral scores in sham and implant groups for forelimb and hindlimb performance over time, consistent with one or more exemplary embodiments of the present disclosure.

### DETAILED DESCRIPTION OF THE INVENTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein is disclosed an exemplary method and system for optical stimulation of target cells in a living body, such as a human or an animal. In one general aspect, the present disclosure may describe a system for optical stimulation of cells in a living body. In an exemplary embodiment, an exemplary system may include an optically stimulating mechanism where an optical stimulation of a plurality of target cells of a target region in an exemplary living body may be done utilizing one or more processors. In an exemplary embodiment, an exemplary system may be utilized for optically stimulation of a plurality of target neurons in an exemplary living body. In an exemplary embodiment, an exemplary system may be utilized for cellular modulation via for example optogenetics to control protein expression. In an exemplary embodiment, an exemplary system may be utilized for neurostimulation of an exemplary plurality of target cells. Moreover, an exemplary system may further include a time programming mechanism. In an exemplary embodiment, an exemplary time programming mechanism may be capable of autonomous starting and ceasing stimulation of cells using predetermined time schedules without a need for external control and manipulation. In addition, an exemplary system may further include a heat controlling mechanism. In an exemplary embodiment, an exemplary heat controlling mechanism may measure a temperature of an exemplary target region and/or a temperature of one or more parts of an exemplary system and perform a process to adjust an exemplary measured temperature at a safe and desired range. Additionally, an exemplary system may further include a wirelessly recharging mechanism. In an exemplary embodiment, an exemplary wirelessly recharging mechanism may be capable of autonomous recharging a power source of an exemplary system without a need for an external connection to a charger device or exchanging an exemplary power source (e.g., a battery). Furthermore, an exemplary system may further include an electrical recording mechanism. In an exemplary embodiment, an exemplary electrical recording mechanism may be utilized to measure and record an electrical parameter in an exemplary target region and calculate and detect cell's behavior of an exemplary target region at different states. Also, an exemplary system may further include a drug delivery mechanism. In an exemplary embodiment, an exemplary drug delivery mechanism may include delivering a therapeutical substance into an exemplary target region and releasing an exemplary therapeutical substance there into.

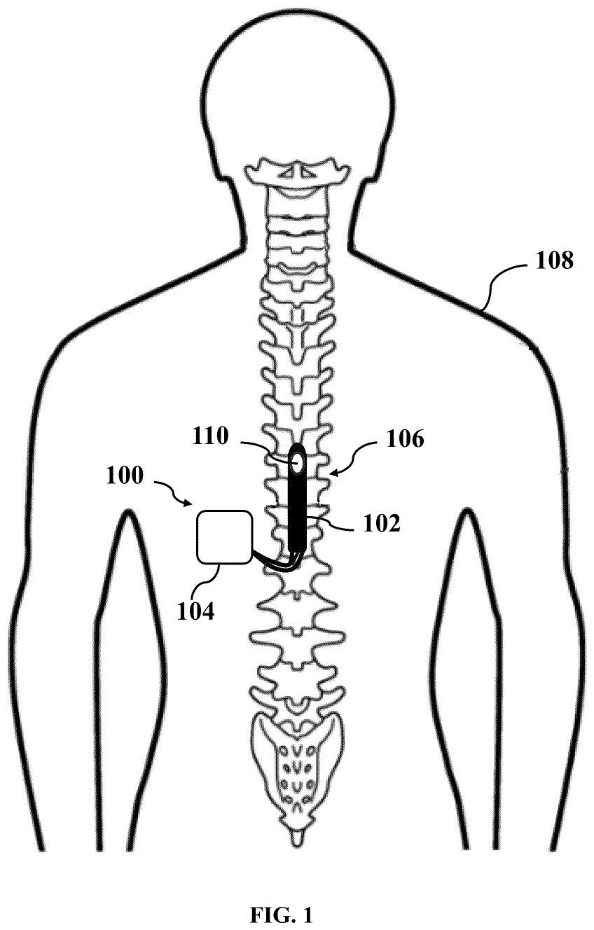

FIG. 1 schematically shows a system 100 for optical neurostimulation implanted in a living body 108, consistent with one or more exemplary embodiments of the present disclosure. In an exemplary embodiment, system 100 may be utilized for optical neurostimulation of target region 106 of living body 108. In an exemplary embodiment, target region 106 may include a portion or whole of at least one of spinal cord, brain, peripheral nerve, heart, eye, muscle tissue, auditory system, and combinations thereof. As exemplary shown in FIG. 1, system 100 may be utilized for optical neurostimulation of spinal cord. In an exemplary embodiment, system 100 may include one or more probes 102 and a microdevice 104 coupled together. In an exemplary embodiment, one or more probes 102 may be implanted in the vicinity of target region 106 and microdevice 104 may be implanted at a location under skin of living body 108 or over skin of living body 108. In an exemplary embodiment, one or more probes 102 and microdevice 104 may be subcutaneously implanted under skin of living body 108. In an exemplary embodiment, one or more probes 102 may include a light emitting element 110 placed in the vicinity of target region 106 so that a light beam drived/delivered by microdevice 104 and generated by light emitting element 110 may be penetrated into target region 106.

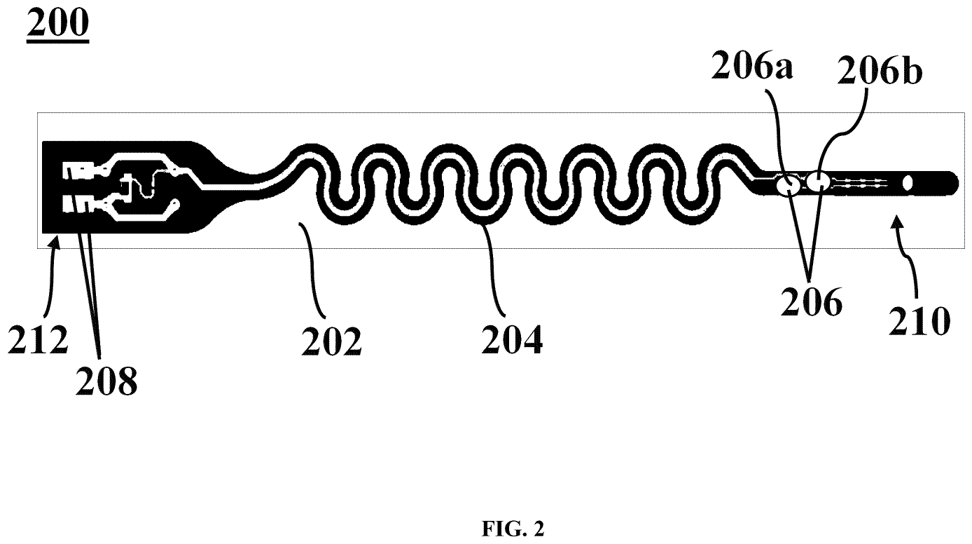

FIG. 2 schematically shows an exemplary probe 200 of one or more probes 102, consistent with one or more exemplary embodiments of the present disclosure. In an exemplary embodiment, probe 200 may include an elastic strip 202, a wire 204, and a plurality of light emitting elements 206. In an exemplary embodiment, elastic strip 202 may include a piece of a soft flexible biocompatible material. In an exemplary embodiment, elastic strip 202 may include a strip of a soft flexible biocompatible polymer. In an exemplary embodiment, elastic strip 202 may include a strip of a soft flexible biocompatible thermoplastic polymer. In an exemplary embodiment, elastic strip 202 may include a strip of at least one of polyimide, Parylene-C, Polydimethylsiloxane (PDMS), Polyurethane, Polyethylene Terephthalate, Polyethylene Naphthalate, a soft biocompatible rubber (e.g., Ecoflex), and combinations thereof. In an exemplary embodiment, elastic strip 202 may include two or more layers of an exemplary soft flexible biocompatible material and wire 204 may be placed between two layers of an exemplary two or more layers of elastic strip 202. In an exemplary embodiment, elastic strip 202 and wire 204 may be stretchable and may have variable lengths. In an exemplary embodiment, wire 204 may include an electrically conductive wire. In an exemplary embodiment, wire 204 may be pressed between two layers of an exemplary two or more layers of elastic strip 202. In an exemplary embodiment, a length of probe 200 which may be approximately equal to a length of either wire 204 or elastic strip 202 may depend on at least one of size of living body 108, a location of target region 106, dimensions of target region 106, and combinations thereof. In an exemplary embodiment, elastic strip 202 may include a soft flexible biocompatible stretchable strip with minimum of 100's of Pa elastic modulus. In an exemplary embodiment, elastic strip 202 may include a soft flexible biocompatible stretchable strip with a length in a range of 0.01 cm to 50 cm and a width in a range of 300 μm to 10 cm. In an exemplary embodiment, wire 204 may include a serpentine-shaped wire. In an exemplary embodiment, wire 204 may have a length in a range of 0.01 cm to 50 cm. In an exemplary embodiment, each of wire 204 and elastic strip 202 may include a stretchable length up to 70 percent of an initial length, respectively. In an exemplary embodiment, probe 200 may be adhered to a location in the vicinity of target region 106 or in contact with target region 106 using a biocompatible paste. In an exemplary embodiment, a longer length of probe 200 (for example, near to maximum length of about 50 cm) may allow for using probe 200 in large animals or humans and/or for connection between different tissues/parts of body, for example, gut-brain axis, which may require a longer probe.

In an exemplary embodiment, plurality of light emitting elements 206 may be mounted on wire 204 along length of wire 204. In an exemplary embodiment, each light emitting element 206a of plurality of light emitting elements 206 may be mounted at a location of at least one of a tip 210 of wire 204, any location along wire 204, and combinations thereof. In an exemplary embodiment, each light emitting element 206a of plurality of light emitting elements 206 may be attached to wire 204 using at least one of a solder paste, a solder metal, and combinations thereof. In an exemplary embodiment, each two light emitting elements 206a and 206b of plurality of light emitting elements 206 may be mounted on wire 204 in series or parallel relation. In an exemplary embodiment, each two light emitting elements 206a and 206b may be arranged in series or parallel apart from each other within a distance of more than 100 μm along wire 204. In an exemplary embodiment, each two light emitting elements 206a and 206b may be arranged in series or parallel apart from each other within a distance of more than 200 μm along wire 204 without any limitations to a maximum distance there between. In an exemplary embodiment, a number and arrangement of plurality of light emitting elements 206 may be selected and designed upon a location and dimensions of target region 106, and a type of stimulation needed. In an exemplary embodiment, elastic strip 202 may be made of a transparent material; allowing for passing light of plurality of light emitting elements 206 there through without any obstacle. In an exemplary embodiment, up to about 30 light emitting elements may be mounted on every 1 cm² of surface area of probe 200. In an exemplary embodiment, plurality of light emitting elements 206 with different wavelengths may be used for simultaneous or sequential activation or inhibition of different cell types via optical stimulation.

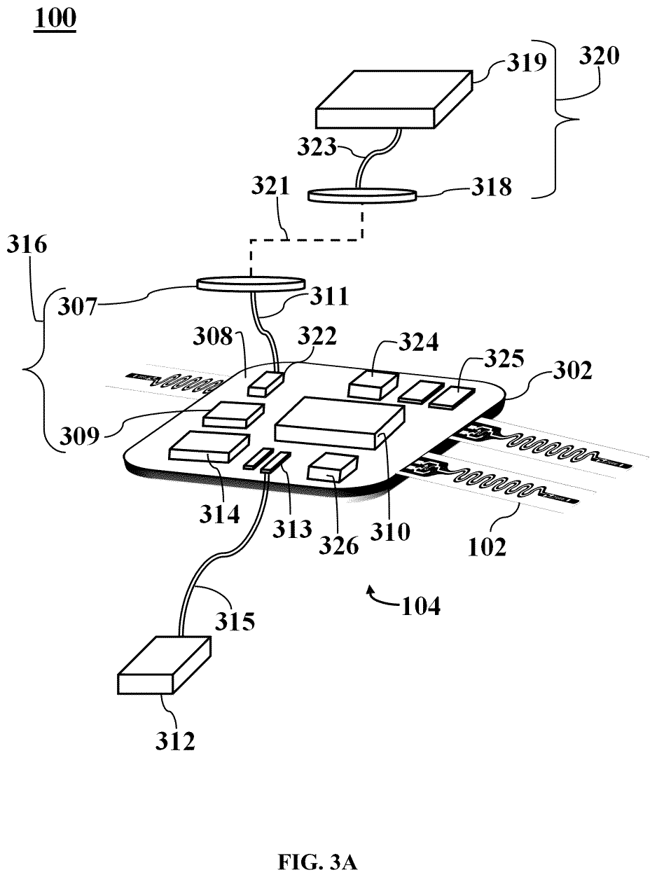

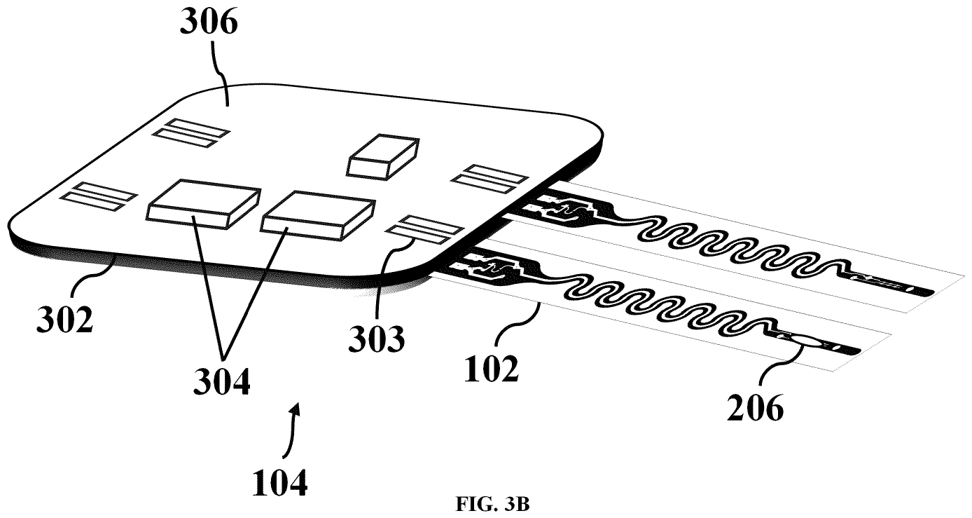

FIG. 3A schematically shows system 100 for optical neurostimulation of target region 106 in living body 108 illustrating a top view of microdevice 104, consistent with one or more exemplary embodiments of the present disclosure. In an exemplary embodiment, system 100 may include one or more probes 102 and microdevice 104. In an exemplary embodiment, microdevice 104 may include a substrate 302 where one or more probes 102 and parts of microdevice 104 may be attached or adhered thereto as illustrated in FIGS. 3A and 3B. In an exemplary embodiment, substrate 302 may include a printed circuit board (PCB). In an exemplary embodiment, substrate 302 may include a piece of a soft flexible biocompatible material. In an exemplary embodiment, substrate 302 may include a piece of a soft flexible biocompatible polymer. In an exemplary embodiment, substrate 302 may include a piece of a soft flexible biocompatible thermoplastic polymer. In an exemplary embodiment, substrate 302 may include a piece of at least one of polyimide, a fiberglass-reinforced epoxy-laminated material, Polyurethane, Polyethylene Terephthalate, Polyethylene Naphthalate, and combinations thereof. In an exemplary embodiment, substrate 302 may include a flat board with a length in a range of 5 mm to 20 mm and a width in a range of 5 mm to 20 mm. In an exemplary embodiment, substrate 302 may include a flat board with a length of 15 mm and a width of 15 mm. In an exemplary embodiment, such small size of substrate 302 and consequently, a small size of microdevice 104 may allow for simple and safe implantation of microdevice 104 in living body 108, specifically, under skin of living body 108. In an exemplary embodiment, substrate 302 may have laser cut smooth edges allowing for preventing damage to a tissue or skin of living body 108 when microdevice 104 is placed in contact thereto.

FIG. 3B schematically shows a bottom view of microdevice 104 in connection with one or more probes 102, consistent with one or more exemplary embodiments of the present disclosure. In an exemplary embodiment, microdevice 104 may include one or more light drivers 304 attached onto substrate 302. In an exemplary embodiment, one or more light drivers 304 may be attached to a bottom surface 306 of substrate 302. In an exemplary embodiment, each light driver of one or more light drivers 304 may be electrically coupled to one or more light emitting elements of plurality of light emitting elements 206. In an exemplary embodiment, a “light driver”, as used herein, may refer to a light emitting element′ driver or a light driver for a one or more light emitting elements of plurality of light emitting elements 206. In an exemplary embodiment, an exemplary light driver may generate a waveform in form of an electrical voltage or current which may be delivered to an exemplary light emitting element, and consequently, a light beam may be generated by an exemplary light emitting element and emitted from an exemplary light emitting element. In an exemplary embodiment, each light driver of one or more light drivers 304 may deliver light through one or more light emitting elements of plurality of light emitting elements 206. In an exemplary embodiment, each light driver of one or more light drivers 304 may generate an accurate voltage on an exemplary light emitting element of plurality of light emitting elements 206 to produce a light beam emitted by an exemplary light emitting element with a desired intensity, timing, frequency, etc. In an exemplary embodiment, each light driver of one or more light drivers 304 may be utilized to generate a programmable electric current and/or voltage waveform to drive an exemplary light emitting element of plurality of light emitting elements 206. In an exemplary embodiment, each light emitting element of plurality of light emitting elements 206 may include a light generator element. In an exemplary embodiment, each light emitting element of plurality of light emitting elements 206 may include a light emitting diode (LED). In an exemplary embodiment, each light driver of one or more light drivers 304 may include a light emitting diode (LED) driver.

In an exemplary embodiment, each light driver of one or more light drivers 304 may be utilized to drive one or more light emitting elements of plurality of light emitting elements 206 to generate an exemplary light beam with a specific set of characteristics. In an exemplary embodiment, each light driver of one or more light drivers 304 may be utilized to deliver light through one or more light emitting elements of plurality of light emitting elements 206 to generate an exemplary light beam with a specific set of characteristics. In an exemplary embodiment, an exemplary specific set of characteristics of an exemplary light beam generated by one or more light emitting elements of plurality of light emitting elements 206 may include a predetermined set of characteristics of an exemplary light beam. In an exemplary embodiment, an exemplary specific set of characteristics may include a range of wavelength (a color) of an exemplary light beam, a frequency of an exemplary light beam, an intensity (a power) of an exemplary light beam, a time duration of emitting an exemplary light beam, and combinations thereof. In an exemplary embodiment, each light driver of one or more light drivers 304 may be utilized to drive one or more light emitting elements of plurality of light emitting elements 206 to generate an exemplary light beam with a wavelength in at least one range of a visible range of 380 nm to 750 nm, an ultraviolet (UV) range of 10 nm to 380 nm, an infrared (IR) range of 750 nm to 1 mm, and combinations thereof. In an exemplary embodiment, each light driver of one or more light drivers 304 may be utilized to drive one or more light emitting elements of plurality of light emitting elements 206 to generate an exemplary light beam with a frequency in a range of 0.001 Hz to 2 MHz. In an exemplary embodiment, each light driver of one or more light drivers 304 may be utilized to drive one or more light emitting elements of plurality of light emitting elements 206 to generate an exemplary light beam with an intensity in a range of 0 W/cm² to 6.95 W/cm². In an exemplary embodiment, each light driver of one or more light drivers 304 may be utilized to drive one or more light emitting elements of plurality of light emitting elements 206 to generate an exemplary light beam in a time duration in a range of 0 seconds to one or more months.

Referring to FIG. 3B, microdevice 104 may further include one or more probe connection ports 303 where one or more probes 102 may be plugged into, respectively. In an exemplary embodiment, one or more probe connection ports 303 may be embedded in substrate 302 in a plurality of different directions; allowing for simultaneously stimulation of target region 106 in various directions with different exemplary specific set of characteristics using more than one probes of one or more probes 102. Furthermore, one or more probe connection ports 303 in an exemplary plurality of different directions may allow for simultaneously stimulation of more than one exemplary target region in living body 108 using more than one probes of one or more probes 102. In an exemplary embodiment, using more than one probe may allow for modulation/sensing of different body parts simultaneously or separately, controlled together or individually. In an exemplary embodiment, exemplary two or more probes may be bundle up to; allowing for simultaneously affecting different types of cells/neurons through different optical wavelengths.

Referring to FIG. 3A, microdevice 104 may further include a processing unit 310 attached onto substrate 302. In an exemplary embodiment, processing unit 310 may include a microcontroller (MCU). In an exemplary embodiment, processing unit 310 may be attached onto a top surface 308 of substrate 302. In an exemplary embodiment, processing unit 310 may be electrically coupled to one or more light drivers 304. In an exemplary embodiment, processing unit 310 may utilize and control one or more light drivers 304 to perform optically stimulation of a plurality cells of target region 106 using plurality of light emitting elements 206. In an exemplary embodiment, processing unit 310 may include a memory and a processor. In an exemplary embodiment, memory may have processor-readable instructions stored therein and processor may be capable of accessing an exemplary memory and execute exemplary processor-readable instructions. In an exemplary embodiment, an exemplary processor may perform a method when exemplary processor-readable instructions are executed by an exemplary processor.

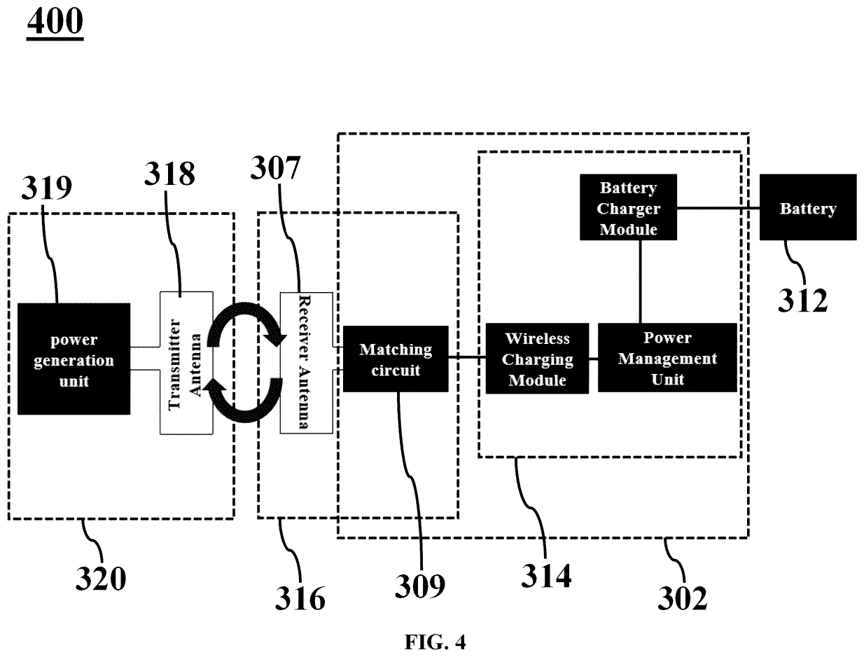

In an exemplary embodiment, microdevice 104 may include an ultra-low energy consuming device with a required power in a range of 360 nW to 160 mW. In an exemplary embodiment, system 100 may further include a wirelessly recharging mechanism for power-needed elements of system 100. In an exemplary embodiment with reference to FIG. 3A, an exemplary wirelessly recharging mechanism may include a rechargeable battery 312 coupled to microdevice 104, a wireless battery charging module 314 attached onto substrate 302, a wireless power receiver 316, and a wireless power transmitter 320. In an exemplary embodiment, rechargeable battery 312 may supply a power consumed by microdevice 104. Furthermore, FIG. 4 schematically shows a block diagram 400 of battery recharging circuit, consistent with one or more exemplary embodiments of the present disclosure.

Referring to FIGS. 3A and 4, rechargeable battery 312 may be attached to substrate 302 at battery connection port 313 via connecting line 315. In an exemplary embodiment, connecting line 315 may include a soft stretchable electrically conductive line; allowing for implanting rechargeable battery 312 either in the vicinity of target region 106 or far from. In an exemplary embodiment, connecting line 315 may be capable of stretching up to 70%. In an exemplary embodiment, rechargeable battery 312 may be subcutaneously placed under skin of living body 108 in the vicinity of target region 106 and microdevice 104. In an exemplary embodiment, rechargeable battery 312 may be subcutaneously placed under skin of living body 108 away from target region 106 and microdevice 104.

In an exemplary embodiment, wireless power receiver 316 may include a receiver antenna 307 and a matching circuit 309. In an exemplary embodiment, matching circuit 309 may tune wireless power reception frequency range, ensuring maximum power transfer between wireless power transmitter and receiver on substrate 302. In an exemplary embodiment, receiver antenna 307 may be coupled to matching circuit 309 on microdevice 104 via a connection between receiver antenna 307 and microdevice 104. In an exemplary embodiment, receiver antenna 307 may be connected to microdevice 104 in tethered fashion. In an exemplary embodiment, receiver antenna 307 may be connected to microdevice 104 by connecting receiver antenna 307 to an antenna connection 322 embedded on substrate 302 via connecting line 311. In an exemplary embodiment, connecting line 311 may include an electrically conductive wire. In an exemplary embodiment, connecting line 311 may include a soft stretchable electrically conductive line; allowing for implanting receiver antenna 307 either in the vicinity of target region 106 or far from. In an exemplary embodiment, connecting line 311 may be capable of stretching up to 70%. In an exemplary embodiment, receiver antenna 307 may be subcutaneously placed under skin of living body 108 in the vicinity of target region 106 and microdevice 104. In an exemplary embodiment, receiver antenna 307 may be placed on microdevice 104. In an exemplary embodiment, receiver antenna 307 may be subcutaneously placed under skin of living body 108 away from target region 106 and microdevice 104. In an exemplary embodiment, microdevice 104, one or more probes 102, receiver antenna 307, and rechargeable battery 312 may be subcutaneously placed under skin of living body 108 via a surgery and cut area may be sutured.

In an exemplary embodiment, wireless power transmitter 320 may include a power generation unit 319 and a transmitter antenna 318. In an exemplary embodiment, transmitter antenna 318 may be wirelessly coupled to receiver antenna 307. In an exemplary embodiment, transmitter antenna 318 may be coupled to receiver antenna 307 through a magnetic resonant connection. In an exemplary embodiment, receiver antenna 307 may receive a signal/field generated by power generation unit 319 and sent by transmitter antenna 318. In an exemplary embodiment, a wireless communication between transmitter antenna 318 and receiver antenna 307 may be used to transfer a power generated by power generation unit 319 to rechargeable battery 312. In an exemplary embodiment, transmitter antenna 318 and power generation unit 319 may be placed outside living body 108. In an exemplary embodiment, transmitter antenna 318 may be placed at a location over skin of living body 108 in the vicinity of receiver antenna 307. In an exemplary embodiment, transmitter antenna 318 and receiver antenna 307 may be placed in the vicinity of target region 106. In an exemplary embodiment, power generation unit 319 may be placed outside of living body 108 far from transmitter antenna 318. In an exemplary embodiment, power generation unit 319 may be placed over skin of living body 108 in the vicinity of transmitter antenna 318 or far from transmitter antenna 318. In an exemplary embodiment, transmitter antenna 318 and receiver antenna 307 may be coupled/connected together through a wireless magnetic resonant connection 321. In an exemplary embodiment, transmitter antenna 318 may be coupled to power generation unit 319 via an electrically conductive line 323 (e.g., a wired connection). In an exemplary embodiment, wireless battery charging module 314 may be coupled to wireless power receiver 316 and rechargeable battery 312. In an exemplary embodiment, wireless battery charging module 314 may transmit a received power by wireless power receiver 316 to rechargeable battery 312. In an exemplary embodiment, rechargeable battery 312 may be charged by wireless battery charging module 314 utilizing a power transmitted from wireless power transmitter 320 to wireless power receiver 316 at a low frequency range of 100 kHz to 200 kHz. In an exemplary embodiment, an exemplary low frequency range of wireless power transmission may allow for undistorted wave and minimum absorption in living body 108, leading to a long-range transmission of wireless power into skin and/or tissue; thereby, resulting in fast and simple recharge of rechargeable battery 312 with minimum absorption by tissues in living body 108.

In an exemplary embodiment, a wireless communication between transmitter antenna 318 and receiver antenna 307 may further be used for further wireless communications. In an exemplary embodiment, transmitter antenna 318 may be coupled to a near-field communication (NFC) device. In an exemplary embodiment, an exemplary NFC device may be utilized for on-demand modulations when needed. An exemplary on-demand modulation may be done through fully passive communication protocols by an exemplary NFC device. In an exemplary embodiment, an exemplary NFC device may be used for powerless programming on-the-fly of operations and functions performed by microdevice 104. In an exemplary embodiment, modulation parameters may be pre-programmed for an autonomous control by microdevice 104 and also may be changed on-the-fly after implantation of microdevice 104 using an exemplary NFC device via a wireless connection through transmitter antenna 318. In an exemplary embodiment, acquired data from target region 106 by various parts of microdevice 104 and/or one or more probes 102 may be wirelessly transmitted to an outside-the-body module and transmitted back upon analysis to a control module for actuation. Also, an exemplary acquired data may be analyzed in microdevice 104 to control actuations of microdevice 104 and one or more probes 102.

In an exemplary embodiment regarding FIG. 3A, system 100 may further include a time programming mechanism. In an exemplary embodiment, an exemplary time programming mechanism may include a real-time calendar (RTC) and/or clock 324 placed on substrate 302. In an exemplary embodiment, microdevice 104 may further include a quartz crystal 325 adhered onto substrate 302 as shown in FIG. 3A. In an exemplary embodiment, quartz crystal 325 may provide accurate tuning of RTC and/or clock 324. In an exemplary embodiment, RTC and/or clock 324 may be coupled to processing unit 310. In an exemplary embodiment, RTC and/or clock 324 may be utilized by one or more processors of processing unit 310 to autonomous controlling time periods of cells'stimulation in target region 106. In an exemplary embodiment, RTC and/or clock 324 may be utilized to start light delivery through each light emitting element 206a or 206b of plurality of light emitting elements 206 at a first predetermined time and cease light delivery through each light emitting element 206a or 206b of plurality of light emitting elements 206 at a second predetermined time.

In an exemplary embodiment, RTC and/or clock 324 may be utilized for at least one of starting light delivery through each light emitting element of plurality of light emitting elements 206 at a first predetermined time, ceasing light delivery through each light emitting element of plurality of light emitting elements 206 at a second predetermined time, turning on one or more functionalities of microdevice 104, turning off one or more functionalities of microdevice 104, switching to a different predetermined set of characteristics of light delivery at a pre-scheduled time or a time during light delivery, and combinations thereof. In an exemplary embodiment, using RTC and/or clock 324 may allow for having pre-determined functionalities such as turning-on/off whole system 100 for applications such as battery-saving. In an exemplary embodiment, RTC and/or clock 324 may be utilized to apply multiple scenarios of optical stimulation of target region 106 by changing an exemplary predetermined set of characteristics of an exemplary generated light beam delivered through one or more light emitting elements of plurality of light emitting elements 206 and emitted to cells of target region 106. In an exemplary embodiment, RTC and/or clock 324 may be utilized to perform at least one of electrical stimulations, electrical recordings, biosensing recordings, and combinations thereof at specific time-points.

In an exemplary embodiment regarding FIG. 3A, system 100 may further include a heat control mechanism. In an exemplary embodiment, system 100 may further include a temperature sensor 326. In an exemplary embodiment, temperature sensor 326 may be adhered onto substrate 302 or one or more probes 102. In an exemplary embodiment, temperature sensor 326 may be coupled to processing unit 310. In an exemplary embodiment, temperature sensor 326 may be utilized by one or more processors of processing unit 310 to autonomous keeping a temperature of target region 106 and nearby tissues at a safe range. In an exemplary embodiment, microdevice 104 and/or one or more probes 102 and nearby environment may be heated while working; thereby, a temperature of target region 106 and/or microdevice 104 may rise up above a safe temperature of about 40° C. for target region 106. In an exemplary embodiment, temperature sensor 326 may be utilized to measure a temperature of target region 106 at least one of before, during, and after optical stimulation of target region 106. In an exemplary embodiment, an exemplary measured temperature may be compared with a threshold temperature value and one or more processes of a set of processes may be performed by processing unit 310 if an exemplary measured temperature is more than an exemplary threshold temperature value. In an exemplary embodiment, an exemplary threshold temperature value may be a temperature range of about 38° C. to 40° C. In an exemplary embodiment, an exemplary threshold temperature value may be a temperature value of about 40° C. In an exemplary embodiment, one or more characteristics of an exemplary predetermined set of characteristics of an exemplary generated light beam may be changed to reduce an exemplary temperature below an exemplary threshold temperature value. In another exemplary embodiment, generating an exemplary light beam by one or more light emitting elements of plurality of light emitting elements 206 may be ceased temporarily or predominantly to reduce an exemplary temperature below an exemplary threshold temperature value.

In an exemplary embodiment, temperature sensor 326 may be utilized to measure a temperature of microdevice 104. In an exemplary embodiment, an exemplary temperature of microdevice 104 may rise up when rechargeable battery 312 is being wirelessly recharged. In an exemplary embodiment, an exemplary heat control mechanism may be utilized to limit an amount of heat generated by microdevice 104 to avoid damage to target region 106 and/or neighboring tissues to a location where microdevice 104 is implanted. In an exemplary embodiment, temperature sensor 326 may be utilized to measure a temperature of microdevice 104 during recharging rechargeable battery 312. In an exemplary embodiment, an exemplary measured temperature may be compared with an exemplary threshold temperature value. In an exemplary embodiment, an exemplary threshold temperature value may be a temperature range of about 38° C. to 40° C. In an exemplary embodiment, an exemplary threshold temperature value may be a temperature value of about 40° C. In an exemplary embodiment, wireless charging of rechargeable battery 312 may be stopped if an exemplary measured temperature of microdevice 104 is more than an exemplary threshold temperature value; allowing for prevention of incident electromagnetic flux heat-up of microdevice 104.

In an exemplary embodiment, system 100 may further include an electrical recording mechanism. In an exemplary embodiment, system 100 may further include an electrical sensor attached to wire 204 of probe 200. In an exemplary embodiment, an exemplary electrical sensor may be coupled to processing unit 310 via at least one of an electrically conductive connecting line (e.g., wire 204), a wireless connection, and combinations thereof. In an exemplary embodiment, an exemplary wireless connection may include Bluetooth devices or Bluetooth modules, which may be embedded in an exemplary electrical sensor and processing unit 310. In an exemplary embodiment, an exemplary electrical sensor may be utilized to measure an electrical parameter of target region 106 at least one of before, during, and after optical stimulation of target region 106 and send an exemplary measured electrical parameter to processing unit 310. In an exemplary embodiment, an exemplary electrical parameter may include at least one of an electrical current of target region 106, an electrical voltage of target region 106, and combinations thereof. In an exemplary embodiment, an exemplary electrical recording mechanism may include recording of an exemplary electrical parameter at a specific time or during a time period utilizing an exemplary electrical sensor. In an exemplary embodiment, an exemplary electrical recording mechanism may include electrical recording of activity of cells (e.g., neurons) in target region 106. In an exemplary embodiment, an exemplary electrical recording mechanism may include analyzing response of cells (e.g., neurons) to an exemplary applied optical stimulation by system 100 based on an exemplary measured and recorded electrical parameter.

In an exemplary embodiment, system 100 may further include a drug delivery mechanism. In an exemplary embodiment, an exemplary drug delivery mechanism may be utilized to deliver a drug to target region 106 and release there. In an exemplary embodiment, an exemplary drug delivery mechanism may include a drug delivery channel (not illustrated) formed in elastic strip 202 and a drug delivery pump (not illustrated) adhered onto substrate 302. In an exemplary embodiment, elastic strip 202 may have multiple layers and an exemplary drug delivery channel may be formed between two layers of exemplary multiple layers. In an exemplary embodiment, an exemplary drug delivery channel may include one or multiple soft and flexible polymeric microfluidic channels embedded in elastic strip 202. In an exemplary embodiment, an exemplary drug delivery pump may be coupled to processing unit 310. In an exemplary embodiment, an exemplary drug delivery pump may be utilized by processing unit 310 to transfer and release a drug into target region 106 through an exemplary drug delivery channel.

In an exemplary embodiment, system 100 may further include one or more photodetectors (not illustrated) which may be utilized for photometry and detecting/measuring cellular activity. In an exemplary embodiment, exemplary one or more photodetectors may be mounted on probe 200. In an exemplary embodiment, system 100 may further include one or more biomarker sensors (not illustrated) for antibody sensing via at least one of fast and short-term sensing, or chronic sensing. In an exemplary embodiment, exemplary one or more biomarker sensors may be mounted on probe 200. In an exemplary embodiment, antibody sensing may include chronic sensing via for example, a carbon nanotube (CNT) coating on probe 200 for signal amplification of reactive oxygen species (ROS). In an exemplary embodiment, exemplary one or more biomarker sensors may include up to 5 different sensors sensing different biomarkers mounted on each probe 200. In an exemplary embodiment, system 100 may further include one or more impedance sensors may be mounted on probe 200 and respective measurement modules may be added to microdevice 104 by which a level of neural myelination, fat formation/insulation and also blood flow of target region 106 may be measured. In an exemplary embodiment, system 100 may further include one or more electrical stimulation electrodes mounted on probe 200. In an exemplary embodiment, exemplary one or more electrical stimulation electrodes may be utilized to electrically stimulation cells of target region 106. In an exemplary embodiment, a diameter of each electrical stimulation electrode of exemplary one or more electrical stimulation electrodes may be in a range of 1 μm to 100 μm. In an exemplary embodiment, each of one or more photodetectors, one or more biomarker sensors, one or more impedance sensors, and one or more electrical stimulation electrodes may be coupled to processing unit 310 via an electrical connecting line or a wireless connection. In an exemplary embodiment, each of one or more photodetectors, one or more biomarker sensors, one or more impedance sensors, and one or more electrical stimulation electrodes may be pre-programmed using microdevice 104 or post-programmed using an exemplary NFC device in wireless communication with processing unit 310 of microdevice 104.

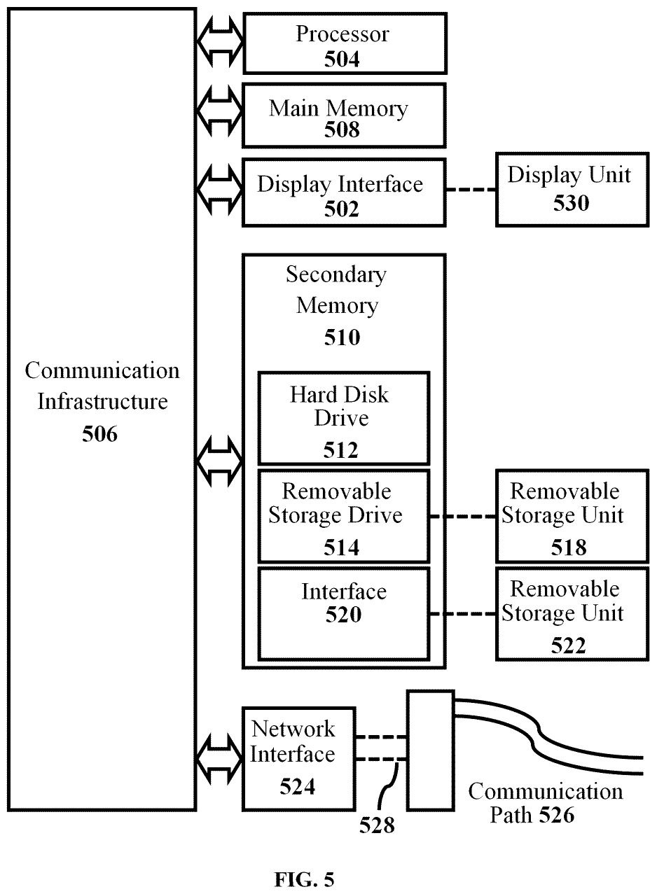

FIG. 5 shows an example computer system 500 in which an embodiment of the present invention, or portions thereof, may be implemented as computer-readable code, consistent with exemplary embodiments of the present disclosure. For example, processes described hereinabove associated with system 100 and/or one or more steps of method 600 described herein below may be implemented in computer system 500 using hardware, software, firmware, tangible computer readable media having instructions stored thereon, or a combination thereof and may be implemented in one or more computer systems or other processing systems. Hardware, software, or any combination of such may embody any of the modules and components in FIGS. 1-4.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the invention is described in terms of this example computer system 500. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 504 may be a special purpose or a general-purpose processor device. As will be appreciated by persons skilled in the relevant art, processor device 504 may also be a single processor in a multi-core/multiprocessor system, such system operating alone, or in a cluster of computing devices operating in a cluster or server farm. Processor device 504 may be connected to a communication infrastructure 506, for example, a bus, message queue, network, or multi-core message-passing scheme.

In an exemplary embodiment, computer system 500 may include a display interface 502, for example a video connector, to transfer data to a display unit 530, for example, a monitor. Computer system 500 may also include a main memory 508, for example, random access memory (RAM), and may also include a secondary memory 510. Secondary memory 510 may include, for example, a hard disk drive 512, and a removable storage drive 514. Removable storage drive 514 may include a floppy disk drive, a magnetic tape drive, an optical disk drive, a flash memory, or the like. Removable storage drive 514 may read from and/or write to a removable storage unit 518 in a well-known manner. Removable storage unit 518 may include a floppy disk, a magnetic tape, an optical disk, etc., which may be read by and written to by removable storage drive 514. As will be appreciated by persons skilled in the relevant art, removable storage unit 518 may include a computer usable storage medium having stored therein computer software and/or data.

In alternative implementations, secondary memory 510 may include other similar means for allowing computer programs or other instructions to be loaded into computer system 500. Such means may include, for example, a removable storage unit 522 and an interface 520. Examples of such means may include a program cartridge and cartridge interface (such as that found in video game devices), a removable memory chip (such as an EPROM, or PROM) and associated socket, and other removable storage units 522 and interfaces 520 which allow software and data to be transferred from removable storage unit 522 to computer system 500.

Computer system 500 may also include a communications interface 524. Communications interface 524 allows software and data to be transferred between computer system 500 and external devices. Communications interface 524 may include a modem, a network interface (such as an Ethernet card), a communications port, a PCMCIA slot and card, or the like. Software and data transferred via communications interface 524 may be in the form of signals, which may be electronic, electromagnetic, optical, or other signals capable of being received by communications interface 524. These signals may be provided to communications interface 524 via a communications path 526. Communications path 526 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 518, removable storage unit 522, and a hard disk installed in hard disk drive 512. Computer program medium and computer usable medium may also refer to memories, such as main memory 508 and secondary memory 510, which may be memory semiconductors (e.g. DRAMs, etc.).

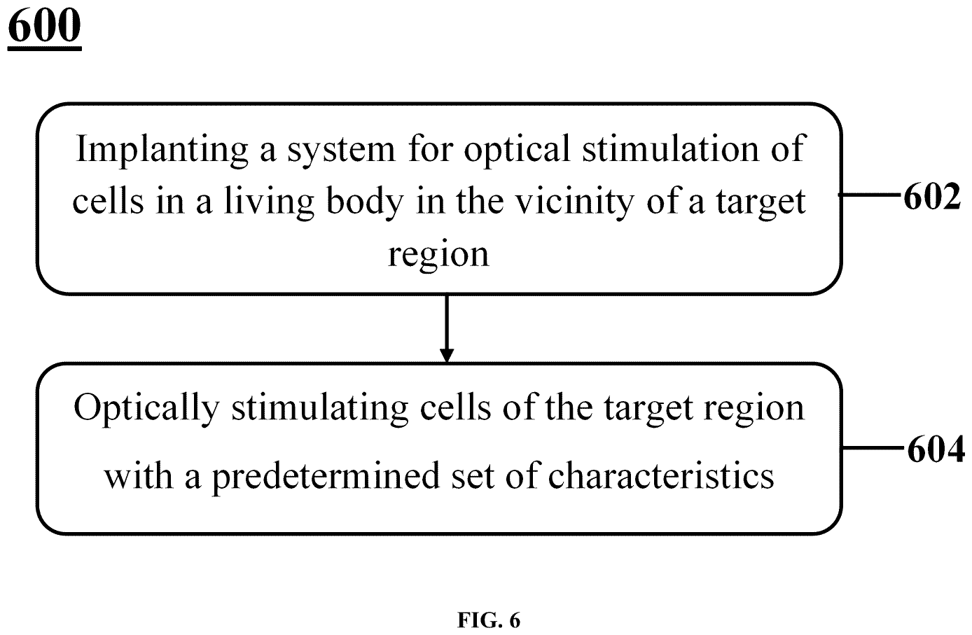

Computer programs (also called computer control logic) are stored in main memory 508 and/or secondary memory 510. Computer programs may also be received via communications interface 524. Such computer programs, when executed, enable computer system 500 to implement different embodiments of the present disclosure as discussed herein. In particular, the computer programs, when executed, enable processor device 504 to implement the processes of the present disclosure, such as the operations described herein above in connection with system 100 and/or operations in method 600 described herein below illustrated by FIGS. 1-4 discussed above and flowchart of FIG. 6 described herein below, respectively. Accordingly, such computer programs represent controllers of computer system 500. Where an exemplary embodiment of method 600 is implemented using software, the software may be stored in a computer program product and loaded into computer system 500 using removable storage drive 514, interface 520, and hard disk drive 512, or communications interface 524.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

In another general aspect, the present disclosure may describe a method for optical stimulation of cells in a living body. In an exemplary embodiment, an exemplary method may be carried out for neurostimulation of a target region in an exemplary living body. In an exemplary embodiment, an exemplary method may be carried out utilizing exemplary system 100 described hereinabove. In an exemplary embodiment, one or more steps of an exemplary method may be implemented utilizing one or more processors. FIG. 6 shows a flowchart of a method 600 for optical stimulation of cells in a living body, consistent with one or more exemplary embodiments of the present disclosure. In an exemplary embodiment, different steps of method 600 may be implemented using system 100. Hence, method 600 may be described herein below in connection with system 100 fully described hereinabove through FIGS. 1-4. In an exemplary embodiment, method 100 may be conducted to at least one of regenerate, grow up, genetically modify, therapeutically treating, and combinations thereof of a plurality of target cells (e.g., neurons) of target region 106. In an exemplary embodiment, method 600 may include implanting system 100 for optical stimulation of cells in living body 108 in the vicinity of target region 106 (step 602) and optically stimulating cells of target region 106 with a predetermined set of characteristics (step 604).

In further detail with respect to step 602, step 602 may include implanting system 100 for optical stimulation of cells in living body 108 in the vicinity of target region 106. In an exemplary embodiment, one or more probes 102 and microdevice 104 of system 100 may be fully implanted at locations inside living body 108 or over skin of living body 108 so that optically stimulation of cells of target region 106 may be done appropriately. In an exemplary embodiment, one or more probes 102 may be put in contact (but not necessarily) with target region 106. In an exemplary embodiment, one or more probes 102 may be put in a distance from target region 106 so that an exemplary light beam of light emitting elements 206 may penetrate into cells of target region 106.

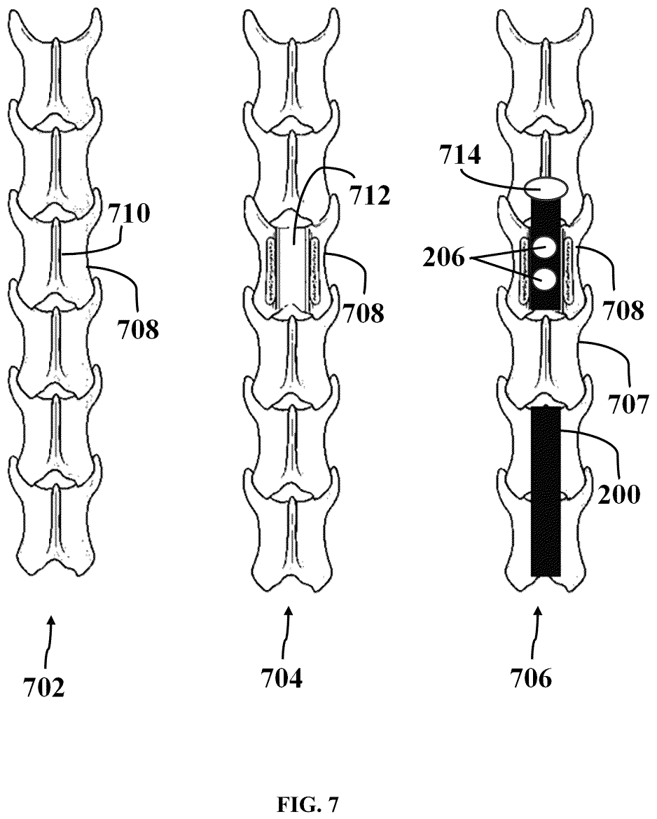

In an exemplary embodiment, method 100 may be conducted to optically stimulation of neurons of a portion of spinal cord as an example of target region 106. FIG. 7 schematically shows an exemplary process 700 of implanting probe 200 in the vicinity of spinal cord of living body 108, consistent with one or more exemplary embodiments of the present disclosure. In an exemplary embodiment, process 700 may include steps of removing a portion 710 of a vertebra 708 so that a portion 712 of spinal cord may become free (parts 702 and 704) and implanting probe 200 on spine by passing a portion of probe 200 trough lamina of a vertebra 707 next to vertebra 708 so that one or more light emitting elements of plurality of light emitting elements 206 may be placed on freely portion 712 of spinal cord (part 706). In an exemplary embodiment, probe 200 may be placed between lamina and spinal cord. In an exemplary embodiment, a biocompatible paste 714 (e.g., a dental cement) may be used to adhere probe 200 on spine. Furthermore, microdevice 104 of system 100 may be implanted under skin of living body 108 in the proximity of probe 200 or far from.

In further detail with respect to step 604, step 604 may include optically stimulating cells of target region 106 (e.g., portion 712 of spinal cord of FIG. 7) with a predetermined set of characteristics. In an exemplary embodiment, optically stimulating cells of target region 106 may include delivering light with a predetermined set of characteristics through each light emitting element of plurality of light emitting elements 206 utilizing one or more light drivers 304. In an exemplary embodiment, delivering light with an exemplary predetermined set of characteristics may include driving one or more light emitting element of plurality of light emitting elements 206 to generate an exemplary light beam with a predetermined magnitude of at least one of a wavelength of an exemplary light beam, a frequency of an exemplary light beam, an intensity of an exemplary light beam, a time duration of emitting an exemplary light beam, and combinations thereof. In an exemplary embodiment, delivering light with an exemplary predetermined set of characteristics may include generating and emitting an exemplary light beam with a wavelength in at least one range of a visible range of 380 nm to 750 nm, an ultraviolet (UV) range of 10 nm to 380 nm, an infrared (IR) range of 750 nm to 1 mm, and combinations thereof. In an exemplary embodiment, delivering light with an exemplary predetermined set of characteristics may include generating and emitting an exemplary light beam with a frequency in a range of 0.001 Hz to 2 MHz. In an exemplary embodiment, delivering light with an exemplary predetermined set of characteristics may include generating and emitting an exemplary light beam with an intensity in a range of 0 W/cm² to 6.95 W/cm². In an exemplary embodiment, delivering light with an exemplary predetermined set of characteristics may include generating/emitting an exemplary light beam with a time duration in a range of 0 seconds to one or more months.

In an exemplary embodiment, optically stimulating cells of target region 106 may further include measuring an electrical parameter of target region 106 at least one of before, during, and after optical neurostimulation of target region 106 using an exemplary electrical sensor coupled to processing unit 310, sending an exemplary measured electrical parameter to processing unit 310, and analyzing electrical behavior of cells of target region 106 based on an exemplary measured electrical parameter. In an exemplary embodiment, an exemplary electrical parameter may include at least one of an electrical current of target region 106, an electrical voltage of target region 106, and combinations thereof.

In an exemplary embodiment, optically stimulating cells of target region 106 may further include scheduling a time program for optically stimulating cells of target region 106 utilizing RTC and/or clock 324. In an exemplary embodiment, scheduling an exemplary time program for optically stimulating cells of target region 106 may include starting light delivery through each light emitting element of plurality of light emitting elements 206 at a first predetermined time using RTC and/or clock 324 and ceasing light delivery through each light emitting element of plurality of light emitting elements 206 at a second predetermined time using RTC and/or clock 324.

In an exemplary embodiment, optically stimulating cells of target region 106 may further include heat controlling of target region 106. In an exemplary embodiment, heat controlling of target region 106 may include measuring a temperature of target region 106 at least one of before, during, and after optical stimulation of target region 106 using temperature sensor 326, comparing an exemplary measured temperature with a threshold temperature value, and performing one or more processes of a set of processes if an exemplary measured temperature is more than an exemplary threshold temperature value. In an exemplary embodiment, an exemplary set of processes may include changing one or more characteristics of an exemplary predetermined set of characteristics and ceasing light delivery through one or more light emitting elements of plurality of light emitting elements 206. In an exemplary embodiment, an exemplary threshold temperature value may be a temperature value in a range of 38° C. to 40° C.

In an exemplary embodiment, optically stimulating cells of target region 106 may further include heat controlling of microdevice 104. In an exemplary embodiment, heat controlling of microdevice 104 may include measuring a temperature of microdevice 104 during recharging rechargeable battery 312 using temperature sensor 326, comparing an exemplary measured temperature with an exemplary threshold temperature value, and ceasing recharging of rechargeable battery if an exemplary measured temperature is more than an exemplary threshold temperature value. In an exemplary embodiment, an exemplary threshold temperature value may be a temperature value in a range of 38° C. to 40° C.

### Example 1: Optical Neuromodulation in Rats

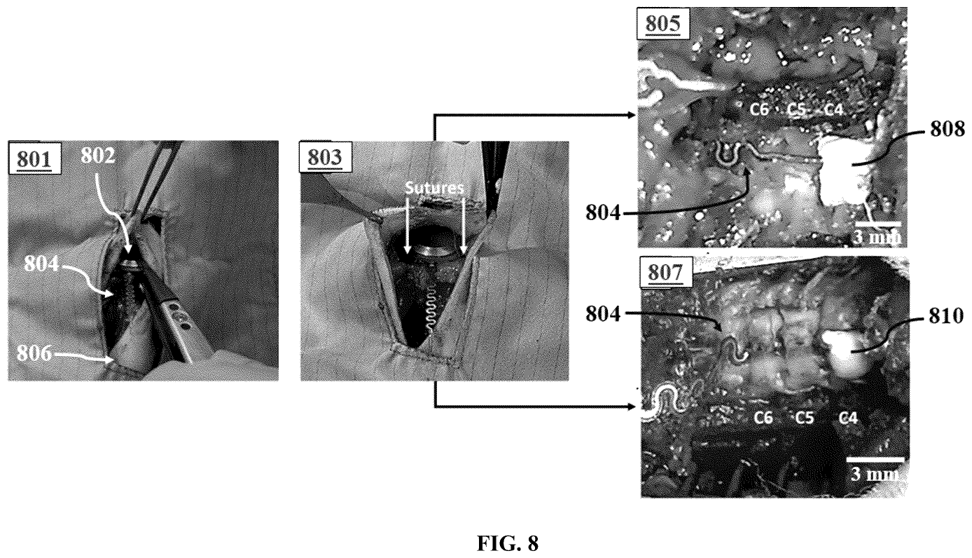

In this example, a system structurally and functionally similar to system 100 as an example of system 100 was designed and fabricated. The system was utilized for optical neuromodulation in rats. FIG. 8 shows device 802 implantation and probe 804 placement in the vicinity of spinal cord of a rat 806, consistent with one or more exemplary embodiments of the present disclosure. As may be seen in part 801, device 802 was placed in subcutaneous pocket of rat 806; then, device 802 was sutured to a musculature tissue of rat 806 (part 803). Probe 804 included a flexible probe with embedded μLEDs on its tip which was activated by an integrated LED driver on device 802. Two different implantations of probe 804 is shown in parts 805 and 807 of FIG. 8. In both situations, tip of probe 804 was secured at C4 lamina with μLEDs hovering over C5 lamina while probe 804's body was implanted differently. In more details, probe 804 may be placed over lamina (part 805) or under lamina (part 807). In part 805, probe 804 was placed on top of spinal cord so that μLEDs of probe 804 was secured above C5 lamina, which had received medial laminectomy. In part 807, probe 804 was passed under the C5 and C6 lamina and raised above C4 lamina through lateral laminectomy; thereby, the μLEDs of probe 804 were again located on top of the spinal cord at C5 but some portion of probe 804 was placed under C6 to reduce mechanical tension. In both parts 805 and 807, tip of probe 804 was cemented at an intact C4 using biocompatible paste pieces 808 and 810, respectively.

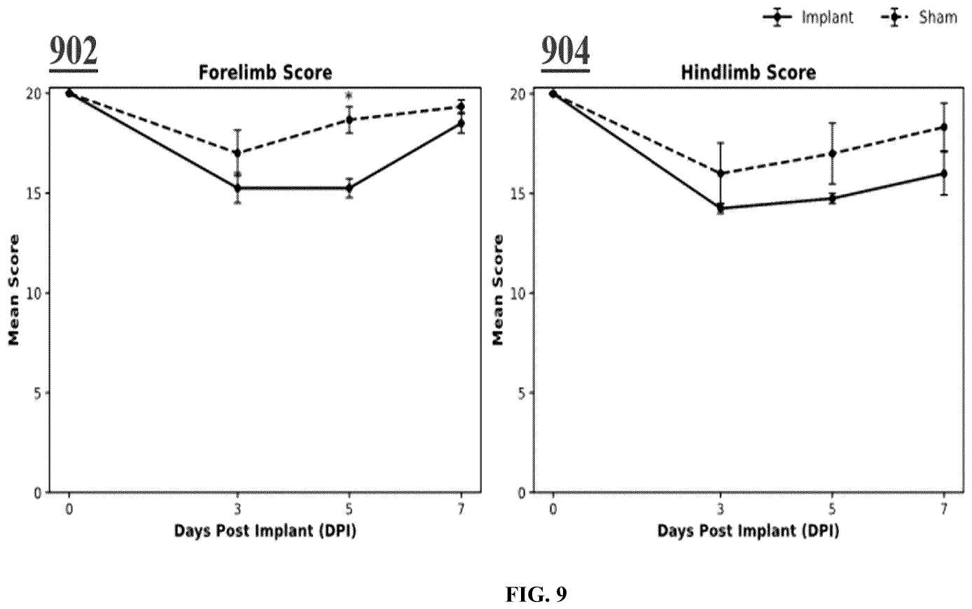

A number of assays were conducted to evaluate motor functions of animals using the Martinez open-field locomotor rating scale. To assess open-field behavior, two trained observers who were unaware of the treatment groups conducted the tests before the operations as well as on days three, five, and seven post-surgery. FIG. 9 shows Martinez open field behavioral scores in sham and implant groups for forelimb (diagram 902) and hindlimb (diagram 904) performance over time, consistent with one or more exemplary embodiments of the present disclosure. The figure legend indicates the sham groups displayed by the dotted line while the implant group is shown by the solid line. The plots illustrate mean behavioral scores for forelimb (diagram 902) and hindlimb (diagram 902) open field assessments across four timepoints of 0 (baseline), 3-, 5-, and 7-days post-implantation (DPI). Error bars represent standard error of the mean (SEM). Significant differences (p<0.05) between the sham and implant groups are indicated with asterisks (*) at specific timepoints. Sham sample size was n=3, and implant was n=4. Following data collection, the non-parametric Mann-Whitney U test was conducted to determine differences at each timepoint for both the forelimb and hindlimb scores between the implant and sham groups. Results analysis indicates a similar forelimb function score in implant and sham groups by day seven (diagram 902). Similarly, there were no statistically significant differences between the groups for the hindlimb scores across all time points (diagram 904). Accordingly, implantation of device 802 and probe 804 in rats'body did not cause any negative effects or muscle weakness or spinal cord injury in rats; thereby, device 802 and probe 804 may be used safely in living bodies.

### INDUSTRIAL APPLICABILITY

An exemplary system disclosed herein may be a fully implantable system for modulation and sensing of tissues that are largely immobile or are under constant tension/release and movement. An exemplary system may be applicable for chronic optical and/or electrical stimulation while also obtaining electrical activity, photometry and biochemical sensing. In the contest of spinal cord, an exemplary system may aid assess the effects of chronic optical stimulation on regeneration of specific types of neurons. In the context of neuroscience, an exemplary system may be used to discover brain-spinal cord neural circuitry. An exemplary system may include a plurality of optical, electrical and/or chemical actuators coupled with photodetectors, chemical biosensors, neural recording and impedance measurement sensors that is fully implantable and is free of tethers and wires external to a living body. An exemplary system further includes one or more flexible probes that can interface and work with different kinds of tissues including fragile and mobile tissues such as spinal cord, peripheral nerves, brain and other organs. The overall size of an exemplary probe and an exemplary microdevice of an exemplary system is small enough to be implanted in rodents as well as larger animals or humans.

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A system for optical stimulation of a target region in a living body, the system comprising:
one or more probes configured to be put in the vicinity of the target region, each respective probe comprising:
an elastic strip comprising two or more layers of a soft flexible biocompatible material;
a wire placed between two layers of the two or more layers of the elastic strip, the wire comprising an electrically conductive wire with a variable length; and
a plurality of light emitting elements mounted on the wire along the length thereof; and
a microdevice configured to be subcutaneously placed under skin of the living body, the microdevice comprising:
a substrate, comprising a piece of a soft flexible biocompatible material;
one or more light drivers attached onto the substrate, each respective light driver coupled to one or more light emitting elements of the plurality of light emitting elements, each respective light driver configured to generate programmable electric current/voltage waveforms to drive the one or more light emitting elements of the plurality of light emitting elements; and
a processing unit attached onto the substrate, the processing unit coupled to the one or more light drivers, the processing unit comprising:
a memory having processor-readable instructions stored therein; and
a processor configured to access the memory and execute the processor-readable instructions, which, when executed by the processor configures the processor to perform a method, the method comprising:
optically stimulating cells of the target region by light delivery, utilizing the one or more light drivers, with a predetermined set of characteristics through each light emitting elements of the plurality of light emitting elements.

2. The system of claim 1, wherein light delivery with the predetermined set of characteristics comprises delivering a light beam with a predetermined magnitude of at least one of a wavelength of the light beam, a frequency of the light beam, an intensity of the light beam, a time duration of light delivery, and combinations thereof.

3. The system of claim 2, wherein light delivery with the predetermined set of characteristics comprises driving each light emitting elements of the plurality of light emitting elements to generate a light beam with a wavelength in at least one range of a visible range of 380 nm to 750 nm, an ultraviolet (UV) range of 10 nm to 380 nm, an infrared (IR) range of 750 nm to 1 mm, and combinations thereof.

4. The system of claim 2, wherein light delivery with the predetermined set of characteristics comprises light delivery with a frequency in a range of 0.001 Hz to 2 MHz.

5. The system of claim 2, wherein light delivery with the predetermined set of characteristics comprises light delivery with an intensity in a range of 0 W/cm² to 6.95 W/cm².

6. The system of claim 2, wherein light delivery with the predetermined set of characteristics comprises light delivery with a time duration in a range of 0 seconds to one or more months.

7. The system of claim 1, wherein the elastic strip comprises a strip of a soft flexible biocompatible polymer with a length in a range of 0.01 cm to 50 cm and a width in a range of 300 μm to 10 cm.

8. The system of claim 1, wherein each of the wire and the elastic strip comprises a stretchable length up to 70 percent of an initial length thereof.

9. The system of claim 1, wherein the wire comprises a serpentine-shaped wire with a length in a range of 0.01 cm to 50 cm.

10. The system of claim 1, wherein each light emitting element of the plurality of light emitting elements comprises a light emitting diode (LED).

11. The system of claim 1, wherein each two light emitting elements of the plurality of light emitting elements are arranged at a location of at least one of a tip of the wire, along the wire, and combinations thereof, in series or parallel relation apart from each other within a distance of more than 100 μm.

12. The system of claim 1, wherein the substrate comprises a piece of a soft flexible biocompatible polymer with a length in a range of 5 mm to 20 mm and a width in a range of 5 mm to 20 mm.

13. The system of claim 1, further comprising an electrical sensor attached to the wire, the electrical sensor configured to:
measure an electrical parameter of the target region at least one of before, during, and after optical stimulation of the target region, the electrical parameter comprising at least one of an electrical current of the target region, an electrical voltage of the target region, and combinations thereof; and
send the measured electrical parameter to the processing unit,
wherein the electrical sensor being coupled to the processing unit/microdevice via at least one of the wires, a wireless connection, and combinations thereof.

14. The system of claim 1, wherein the microdevice further comprises a real-time calendar (RTC) placed on the substrate, the RTC being coupled to the processing unit,
wherein the method further comprises at least one of starting light delivery through each light emitting element of the plurality of light emitting elements at a first predetermined time, ceasing light delivery through each light emitting element of the plurality of light emitting elements at a second predetermined time, turning on one or more functionalities of the microdevice, turning off one or more functionalities of the microdevice, switching to a different predetermined set of characteristics of light delivery at a pre-scheduled time or a time during light delivery, and combinations thereof.

15. The system of claim 1, further comprising a temperature sensor adhered onto the elastic strip of the probe, the temperature sensor being coupled to the processing unit, wherein the method further comprises:
measuring, utilizing the temperature sensor, a temperature of the target region at least one of before, during, and after optical stimulation of the target region;
comparing the measured temperature with a threshold temperature value; and
performing one or more processes of a set of processes responsive to the measured temperature being more than the threshold temperature value, the set of processes comprising:
changing one or more characteristics of the predetermined set of characteristics; and
ceasing light delivery through one or more light emitting elements of the plurality of light emitting elements.

16. The system of claim 1, wherein the microdevice comprises an ultra-low energy consuming device with a required power in a range of 360 nW to 160 mW.

17. The system of claim 1, further comprising a wirelessly power recharging mechanism, comprising:
a rechargeable battery coupled to the microdevice via a soft stretchable electrically conductive connecting line, the rechargeable battery being configured to provide a power of the microdevice, the rechargeable battery being subcutaneously placed under skin of the living body;
a wireless power receiver coupled to the microdevice, the wireless power receiver comprising a receiver antenna connected to the microdevice, the receiver antenna being subcutaneously placed under skin of the living body;
a wireless power transmitter comprising a power generation unit and a transmitter antenna coupled together, the transmitter antenna being wirelessly coupled to the receiver antenna, the transmitter antenna being placed at a location over skin of the living body in the vicinity of the receiver antenna; and
a wireless battery charging module attached onto the substrate, the wireless battery charging module being coupled to the wireless power receiver and the rechargeable battery,
wherein the rechargeable battery is charged by the wireless battery charging module utilizing a power transmitted from the wireless power transmitter to the wireless power receiver at a frequency range of 100 kHz to 200 kHz.

18. The system of claim 17, further comprising a temperature sensor adhered onto the substrate of the microdevice, the temperature sensor being coupled to the processing unit,
wherein the method further comprises:
measuring, utilizing the temperature sensor, a temperature of the microdevice during recharging the rechargeable battery;
comparing the measured temperature with a threshold temperature value;
and
ceasing recharging of the rechargeable battery responsive to the measured temperature being more than the threshold temperature value.

19. The system of claim 1, further comprising a drug delivery mechanism configured to deliver a drug to the target region, the drug delivery mechanism comprising:
a drug delivery channel formed in the elastic strip; and
a drug delivery pump adhered onto the substrate, the drug delivery pump being coupled to the processing unit,
wherein the method further comprises releasing a drug into the target region through the drug delivery channel utilizing the drug delivery pump.

20. The system of claim 1, further comprising at least one of:
one or more photodetectors mounted on the probe, the one or more photodetectors configured to detect and measure cellular activity of the target region;
one or more biomarker sensors mounted on the probe, the one or more biomarker sensors configured to sense an antibody in the target region via at least one of fast sensing, short-term sensing, chronic sensing, and combinations thereof;
one or more impedance sensors mounted on the probe, the one or more impedance sensors configured to measure at least one of a level of neural myelination, fat formation/insulation, blood flow, and combinations thereof in the target region; and
one or more electrical stimulation electrodes mounted on the probe, the one or more electrical stimulation electrodes configured to electrically stimulating cells of the target region.
